(19) United States
(12) Patent Application Publication
(10) Pub. No.: US 2015/0099654 A1
(43) Pub. Date: Apr. 9, 2015

(54) REAL TIME PCR DETECTION OF RESPIRATORY SYNCYTIAL VIRUS

(71) Applicant: VELA OPERATIONS PTE. LTD., Singapore (SG)

(72) Inventors: Boon King Teh, Singapore (SG); Damian Foo, Singapore (SG)

(21) Appl. No.: 14/381,583

(22) PCT Filed: Feb. 28, 2013
(86) PCT No.: PCT/IB2013/051606

§ 371 Date: Aug. 27, 2014

(30) Foreign Application Priority Data
Feb. 29, 2012 (GB) 1203563.0

(51) Int. Cl. C12Q 1/70

(52) U.S. Cl. CPC C12Q 1/701; C12Q 2600/158; C12Q 2600/156
USPC 506/9; 435/5

(57) ABSTRACT

The present invention relates to assays, diagnostic kits and methods for real-time PCR detection of RSV, preferably for the detection of both human pathogens RSV A and RSV B at the same time.

### FIELD OF THE INVENTION

The present invention relates to the detection of Respiratory Syncytial Virus (RSV) using real-time PCR.

### BACKGROUND

Human respiratory Syncytial Virus (RSV) is a virus that infects cells of the respiratory tract and is one of the main causes for lower respiratory tract infections and hospitalizations of infants and children. Currently, no vaccine is available to prevent infections, which are one of the main causes of bronchiolitis and pneumonia in children under one year of age and pneumonia in immuno-compromised patients, e.g. transplant patients or HIV-infected individuals.

As RSV spreads very easily, it is important to identify infected patients as fast as possible and to isolate them from the community, e.g. in hospitals, etc.

RSV infection can be confirmed using antibody-based detection methods, detection of viral RNA using reverse transcriptase PCR and various other methods that are relatively time-consuming such as plaque assay, antigen capture enzyme immune-assay (EIA), enzyme-linked immunosorbent assay and hemaglutination assay or neutralization assays.

There is a need in the art to provide rapid qualitative methods for the detection of RSV in a biological specimen, wherein the methods are as fast as possible and as sensitive as possible, preferably capable of detecting only a few copies of viral RNA in total RNA extracted from a biological sample from a patient, e.g. only in a few femtogram of total extracted RNA (e.g. 100 fg, or preferably 10 fg-100 fg). As RNA isolation or extraction from biological samples can affect the integrity of these molecules, it is another objective to provide an assay for the detection of RSV that comprises an extraction control to exclude false negative results, but at the same time maintain the above mentioned high sensitivity. These objectives are achieved with the new assay, the kits, compositions and methods disclosed herein below.

### SUMMARY

The present invention provides an assay for the detection of Respiratory Syncytial Virus for real-time qualitative PCR.

The present invention also relates to a method for the simultaneous detection of RSV A and RSV B in a biological sample from a patient, comprising:
a) providing a biological sample from a patient;
b) extracting viral RNA from the biological sample;
c) carrying out an RT-PCR with the primers according to the present invention; and
d) detecting amplification products, wherein the presence of an amplification product is indicative of the presence of the virus in the biological sample.

In an alternative method, instead of carrying a RT-PCR, the method comprises a step of reverse-transcription and a step of PCR amplification.

The present invention further concerns the use of a set of nucleic acids according to the present invention for simultaneously detecting RSV A and RSV B.

It further concerns a method of simultaneously detecting RSV A and/or RSV B by using a set of nucleic acids according to the present invention.

In addition, it concerns sets of nucleic acids according to the present invention for preparing a diagnostic kit useful for detecting RSV A and/or B. Optionally, the kit further comprises other components such as positive control RNA plus primers and probes to detect the same, DNA polymerase, a reverse-transcriptase, RNase inhibitors, dNTPs and a PCR and/or RT-buffers.

### SHORT SUMMARY OF PREFERRED EMBODIMENTS

Some of the preferred embodiments of the invention are depicted below:
i. A method for the detection of the presence or absence of at least one nucleic acid of Respiratory Syncytial Virus (RSV) in a biological sample, wherein the method comprises conducting real-time PCR. Preferably, the method is suitable to detect RSV RNA in as low as 100 fg total extracted RNA or less, more preferably in only 10-100 fg RNA.
ii. The method according to embodiment (i) further comprising isolating nucleic acids from the biological sample and performing a reverse transcription step.
iii. The method according to any one of embodiments (i) or (ii), wherein primer sets that are specific for RSV A and/or RSV B are used.
iv. The method according to any one of embodiments (i) to (iii), wherein the primers are suitable for amplification of a region of the RSV M gene.
v. The method according to any one of embodiments (i) to (iv), wherein the RSV-specific primer set comprises oligonucleotide sequences set forth in SEQ ID Nos: 1 and 2 or 1 and 3, or complements or homologues thereof.
vi. The method according to any one of embodiments (i) to (v), wherein at least one probe specifically binding to a nucleic acid of RSV A and/or RSV B is used.
vii. The method according to embodiment (vi), wherein the at least one probe is selected from the oligonucleotides set forth in SEQ ID Nos: 4 to 6.
viii. The method according to any one of embodiments (i) to (vii), wherein the primers and/or probes carry a fluorescent moiety.
ix. A method for the diagnosis of an RSV infection comprising performing one of the methods according to any one of the preceding embodiments.
x. A method for monitoring the treatment of RSV infection, said method comprising performing the method according to embodiment (ix) before treatment with at least one anti-viral drug and during and/or after treatment with said anti-viral drug.
xi. An assay for simultaneous detection of at least one nucleic acid of RSV A and RSV B in a biological sample comprising primers specifically hybridizing to nucleic acids derived from said RSV A and/or B, wherein said assay is suitable for real-time PCR.
xii. The assay according to embodiment (xi), wherein the assay comprises primers and/or probes set forth in the preceding embodiments.
xiii. The assay according to embodiment (xii), wherein the assay is adapted for use in a fully automated laboratory.
xiv. A diagnostic composition comprising primers and/or probes set forth in any one of the preceding embodiments.
xv. A diagnostic kit for the detection of RSV A and/or B in a biological sample comprising primers and/or probes set forth in any one of the preceding embodiments, and optionally comprising instructions for use.
xvi. The diagnostic kit according to embodiment (xv), wherein said kit further comprises enzymes, deoxynucleotides, and/or buffers for performing a reverse transcription step and/or a PCR step.
xvii. The diagnostic kit according to any one of embodiments (xv) or (xvi) further comprising reagents for the isolation of nucleic acids from a biological sample.

### SUMMARY

The invention provides for methods of identifying Respiratory Syncytial Virus (RSV) RNA by real-time polymerase chain reaction (PCR) in a biological sample. Primers and probes for detecting RSV are also provided by the invention, as are kits or compositions containing such primers and probes.

Methods of the invention can be used to identify RNA from specimens for diagnosis of RSV infection. The specific primers and probes of the invention that are used in these methods allow for the amplification and monitoring the development of specific amplification products.

In particular an assay for RSV is provided, which allows for simultaneous detection and/or diagnosis of RSV A and RSV B, respectively. The assay is suitable to detect very low amounts of viral RNA, e.g. in about 100 fg of total extracted RNA or less, preferably 10-100 fg of total RNA extracted from a sample.

According to one aspect of the invention, a method for detecting the presence or absence of RSV in a biological sample from an individual is provided. As RSV viruses are RNA viruses, the method comprises a reverse transcription step, at least one cycling step, which includes an amplifying step and a hybridizing step. The amplifying step includes contacting the sample with at least one pair of specific primers to produce an amplification product if an RSV nucleic acid molecule is present in the sample. The hybridization step includes contacting the sample with RSV virus specific probes. In the assays of the present invention several primer pairs are used that are suitable to hybridize to nucleic acids, but not to other nucleic acids of other viruses. As a result of the methods described herein, the amplification and subsequent detection of the target nucleic acids is possible. A pair of RSV primers comprises a first RSV primer and a second RSV primer. Sequences of the primers and the probes of the invention are shown in the sequence listing.

In some aspects of the invention, the primers and/or probes of the invention can be labeled with a fluorescent moiety. Fluorescent moieties for use in real-time PCR detection are known to persons skilled in the art and are available from various commercial sources, e.g. from life technologies™ or other suppliers of ingredients for real-time PCR.

Representative biological samples from the respiratory tract include throat swabs, throat washings, nasal swabs, and specimens from the lower respiratory tract. In addition, the cycling step can be performed on a control sample. A control sample can include the same portion of the RSV nucleic acid molecule. Alternatively, a control sample can include a nucleic acid molecule other than an RSV nucleic acid molecule. One example for a control nucleic acid would be RNA from a different virus that is not pathogenic to humans, e.g. of tobacco mosaic virus (TMV). Control RNA may be added during extraction to check the integrity of extracted RNA. Including an extraction control serves also to detect false negative results.

Cycling steps can be performed on such a control sample using a pair of control primers and a pair of control probes. The control primers and probes are different from RSV primers and probes.

One or more amplifying steps produces a control amplification product. Each of the control probes hybridizes to the control amplification product.

In another aspect of the invention, there are provided articles of manufacture, or kits.

Kits of the invention can include at least one pair of specific primers for the amplification of RSV and at least one probe hybridizing specifically with the amplification products.

Articles of manufacture can include fluorophoric moieties for labeling the primers or probes or the primers and probes are already labeled with donor and corresponding acceptor fluorescent moieties.

The article of manufacture can also include a package insert having instructions thereon for using the primers, probes, and fluorophoric moieties to detect the presence or absence of RSV in a sample.

In another aspect of the invention, there is provided a method for detecting the presence or absence of RSV in a biological sample from an individual. Such a method includes performing at least one cycling step. A cycling step include at least one amplifying step and a hybridizing step. Generally, an amplifying step includes contacting the sample with a pair of primers to produce an amplification product if an RSV nucleic acid molecule is present in the sample. Generally, a hybridizing step includes contacting the sample with an RSV-specific probe. The probe is usually labeled with at least one fluorescent moiety. The presence or absence of fluorescence is indicative of the presence or absence of RSV in said sample.

Amplification generally involves the use of a polymerase enzyme. Suitable enzymes are known in the art, e.g. Taq Polymerase, etc.

In another aspect of the invention, there is provided a method for detecting the presence or absence of RSV in a biological sample from an individual. Such a method includes performing at least one cycling step. A cycling step can include an amplifying step and a dye-binding step. An amplifying step generally includes contacting the sample with a pair of RSV-specific primers to produce an RSV amplification product if an RSV nucleic acid molecule is present in the sample. A dye-binding step generally includes contacting the RSV amplification product with a double-stranded DNA binding dye. The method further includes detecting the presence or absence of binding of the double-stranded DNA binding dye into the amplification product. According to the invention, the presence of binding is typically indicative of the presence of RSV nucleic acid in the sample, and the absence of binding is typically indicative of the absence of RSV nucleic acid in the sample. Such a method can further include the steps of determining the melting temperature between the amplification product and the double-stranded DNA binding dye. Generally, the melting temperature confirms the presence or absence of RSV nucleic acid. Representative double-stranded DNA binding dyes include SYBRGREEN I®, SYBRGOLD®, and ethidium bromide.

In another aspect, the invention allows for the use of the methods described herein to determine whether or not an individual is in need of treatment for RSV.

The invention also provides for the use of the articles of manufacture described herein to determine whether or not an individual is in need of treatment for RSV.

Further, the methods and/or the articles of manufacture described herein can be used to monitor an individual for the effectiveness of a treatment for RSV as well as in epidemiology to monitor the transmission and progression of RSV from individuals to individuals in a population. The methods and/or the articles of manufacture (e.g., kits) disclosed herein can be used to determine whether or not a patient is in need of treatment for RSV.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will be decisive.

The details of one or more embodiments of the invention are set forth in the description below. Other features, objects, and advantages of the invention will be apparent from the description, and from the claims.

### DETAILED DESCRIPTION

According to the present invention, a real-time PCR assay for detecting RSV virus nucleic in a biological sample that is more sensitive and specific than existing assays is described herein.

Primers and probes for detecting RSV infections and articles of manufacture containing such primers and probes are also provided. The increased sensitivity of real-time PCR for detection of RSV as well as the improved features of real-time PCR including sample containment and real-time detection of the amplified product, make feasible the implementation of this technology for routine diagnosis of RSV infections in the clinical laboratory.

The invention provides methods to detect RSV by amplifying, for example, a portion of an RSV nucleic acid derived from the M gene (an example has been deposited at GenBank: AAX23991.1 corresponding to nucleotides 3261 to 4031 of human RSV strain ATCC VR-26, complete genome has been deposited at GenBank under AY911262.1). Nucleic acid sequences from RSV A and B are available, e.g. in public databases, e.g. in GenBank under AY911262.1 (human RSV subtype A) and AY353550.1 (human RSV subtype B), respectively.

Primers and probes can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection, similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 15 to 30 nucleotides in length. Designing oligonucleotides to be used as hybridization probes can be performed in a manner similar to the design of primers, although the members of a pair of probes preferably anneal to an amplification product. As with oligonucleotide primers, oligonucleotide probes usually have similar melting temperatures, and the length of each probe must be sufficient for sequence-specific hybridization to occur but not so long that fidelity is reduced during synthesis. Oligonucleotide probes are generally 15 to 30 nucleotides in length. Primers useful within the context of the present invention include oligonucleotides suitable in PCR reactions for the amplification of nucleic acids derived from nucleic acids coding for the M gene of RSV A and/or B, respectively.

In describing and claiming the present invention, the terminology and definitions hereinbelow are used for the purpose of describing particular embodiments only, and are not intended to be limiting.

As used in this specification and the appended claims, the singular forms “a,” “an,” and “the” include plural referents unless the context clearly dictates otherwise.

Within the context of the present invention, a “multiplex assay” would be for example, a molecular assay that simultaneously screens for RSV A and RSV B.

As used herein, the term “probe” or “detection probe” refers to an oligonucleotide that forms a hybrid structure with a target sequence contained in a molecule (i.e., a “target molecule”) in a sample undergoing analysis, due to complementarity of at least one sequence in the probe with the target sequence. The nucleotides of any particular probe may be deoxyribonucleotides, ribonucleotides, and/or synthetic nucleotide analogs.

The term “primer” or “amplification primer” refers to an oligonucleotide that is capable of acting as a point of initiation for the 5′ to 3′ synthesis of a primer extension product that is complementary to a nucleic acid strand. The primer extension product is synthesized in the presence of appropriate nucleotides and an agent for polymerization such as a DNA polymerase in an appropriate buffer and at a suitable temperature.

As used herein, the term “homology” or “homologue” means that a sequence, e.g. a primer or probe sequence disclosed herein, is essentially identical to the sequence of said primer or probe, but may instead of deoxyribunucleotides comprise corresponding ribonucleotides or synthetic analogues. Homologs of a given sequence hybridize to the same target sequence and permit amplification of a target region in a gene of interest, or they bind to target regions as probes and may be detected, e.g. because they carry fluorescent moieties. Identical sequences correspond to the sequences of the primers and/or probes of the present invention, but they may not be 100% identical, e.g. when one or more residues of the total sequences have been replaced by another residue, or because the 5′ or 3′ ends of the primers/probes disclosed herein have been shortened or lengthened. Such primers/probes maintain their capability of hybridizing with a target region and permitting amplification or detection of said target region.

As used herein, the term “target amplification” refers to enzyme-mediated procedures that are capable of producing billions of copies of nucleic acid target. Examples of enzyme-mediated target amplification procedures known in the art include PCR.

Within the context of the present invention, the nucleic acid “target” is a nucleic acid sequence region of the M gene of RSV A and/or RSV B.

The most widely used target amplification procedure is PCR, first described for the amplification of DNA by Mullis et al. in U.S. Pat. No. 4,683,195 and Mullis in U.S. Pat. No. 4,683,202 and is well known to those of ordinary skill in the art. Where the starting material for the PCR reaction is RNA, complementary DNA (“cDNA”) is made from RNA via reverse transcription. A PCR used to amplify RNA products is referred to as reverse transcriptase PCR or “RT-PCR.” In the PCR technique, a sample of DNA is mixed in a solution with a molar excess of at least two oligonucleotide primers of that are prepared to be complementary to the 3′ end of each strand of the DNA duplex; a molar excess of nucleotide bases (i.e., dNTPs); and a heat stable DNA polymerase, (preferably Taq polymerase), which catalyzes the formation of DNA from the oligonucleotide primers and dNTPs. Of the primers, at least one is a forward primer that will bind in the 5′ to 3′ direction to the 3′ end of one strand of the denatured DNA analyte and another is a reverse primer that will bind in the 3′ to 5′ direction to the 5′ end of the other strand of the denatured DNA analyte. The solution is heated to 94-96° C. to denature the double-stranded DNA to single-stranded DNA. When the solution cools down and reaches the so-called annealing temperature, the primers bind to separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. As the sequence being amplified doubles after each cycle, a theoretical amplification of a huge number of copies may be attained after repeating the process for a few hours; accordingly, extremely small quantities of DNA may be amplified using PCR in a relatively short period of time.

Where the starting material for the PCR reaction is RNA, as in the case of RSV virus nucleic acids, complementary DNA (“cDNA”) is synthesized from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. A PCR used to amplify RNA products is referred to as reverse transcriptase PCR or “RT-PCR.”

The terms “real-time PCR” and “real-time RT-PCR,” refer to the detection of PCR products via a fluorescent signal generated by the coupling of a fluorogenic dye molecule and a quencher moiety to the same or different oligonucleotide substrates. Examples of commonly used probes are TAQMAN® probes, Molecular Beacon probes, SCORPION® probes, and SYBR® Green probes. Briefly, TAQMAN® probes, Molecular Beacons, and SCORPION® probes each have a fluorescent reporter dye (also called a “fluor”) attached to the 5′ end of the probes and a quencher moiety coupled to the 3′ end of the probes. In the unhybridized state, the proximity of the fluor and the quencher molecules prevents the detection of fluorescent signal from the probe; during PCR, when the polymerase replicates a template on which a probe is bound, the 5′-nuclease activity of the polymerase cleaves the probe thus, increasing fluorescence with each replication cycle. SYBR Green® probes binds double-stranded DNA and upon excitation emit light; thus as PCR product accumulates, fluorescence increases. In the context of the present invention, the use of TAQMAN® probes is preferred.

The terms “complementary” and “substantially complementary” refer to base pairing between nucleotides or nucleic acids, such as, for instance, between the two strands of a double-stranded DNA molecule or between an oligonucleotide primer and a primer binding site on a single-stranded nucleic acid to be sequenced or amplified. Complementary nucleotides are, generally, A and T (or A and U), and G and C. Within the context of the present invention, it is to be understood that the specific sequence lengths listed are illustrative and not limiting and that sequences covering the same map positions, but having slightly fewer or greater numbers of bases are deemed to be equivalents of the sequences and fall within the scope of the invention, provided they will hybridize to the same positions on the target as the listed sequences. Because it is understood that nucleic acids do not require complete complementarity in order to hybridize, the probe and primer sequences disclosed herein may be modified to some extent without loss of utility as specific primers and probes. Generally, sequences having homology of about 80%, 85%, 90% or 95% or more fall within the scope of the present invention. As is known in the art, hybridization of complementary and partially complementary nucleic acid sequences may be obtained by adjustment of the hybridization conditions to increase or decrease stringency, i.e., by adjustment of hybridization temperature or salt content of the buffer.

The term “hybridizing conditions” is intended to mean those conditions of time, temperature, and pH, and the necessary amounts and concentrations of reactants and reagents, sufficient to allow at least a portion of complementary sequences to anneal with each other. As is well known in the art, the time, temperature, and pH conditions required to accomplish hybridization depend on the size of the oligonucleotide probe or primer to be hybridized, the degree of complementarity between the oligonucleotide probe or primer and the target, and the presence of other materials in the hybridization reaction admixture. The actual conditions necessary for each hybridization step are well known in the art or can be determined without undue experimentation.

The term “label” as used herein refers to any atom or molecule that can be used to provide a detectable (preferably quantifiable) signal, and that can be attached to a nucleic acid or protein via a covalent bond or noncovalent interaction (e.g., through ionic or hydrogen bonding, or via immobilization, adsorption, or the like). Labels generally provide signals detectable by fluorescence, chemiluminescence, radioactivity, colorimetry, mass spectrometry, X-ray diffraction or absorption, magnetism, enzymatic activity, or the like. Examples of labels include fluorophores, chromophores, radioactive atoms, electron-dense reagents, enzymes, and ligands having specific binding partners.

As used herein, the term “sample” as used in its broadest sense to refer to any biological sample from any human or veterinary subject that may be tested for the presence or absence of one or more RSV-specific nucleic acids. The samples may include, without limitation, tissues obtained from any organ, such as for example, lung tissue; and fluids obtained from any organ such as for example, blood, plasma, serum, lymphatic fluid, synovial fluid, cerebrospinal fluid, amniotic fluid, amniotic cord blood, tears, saliva, and nasopharyngeal washes.

The term “patient” as used herein is meant to include both human and veterinary patients.

The amplification primers and detection probes of the present invention are set forth in the sequence listing.

In one aspect of the invention, there is provided a method for detection of RSV in a sample comprising the steps of obtaining a biological sample from a patient; isolating nucleic acid from the sample; amplifying the nucleic acid, wherein the nucleic acid is amplified and detected with amplification primers and detection probes selected from the group depicted in the sequence listing.

In another aspect of the invention, there is provided a method for detection of RSV in a sample comprising the steps of obtaining a tissue sample from a patient; extracting nucleic acids from the sample; amplifying the nucleic acid, wherein the RNA is amplified and detected with amplification primers and detection probes as depicted in the sequence listing.

In one embodiment of the invention, the nucleic acid is selected from RNA and DNA. When the nucleic acid is RNA, it is amplified using real time RT-PCR. When the nucleic acid is DNA, it is amplified using real time PCR.

In another embodiment of the invention, the sample is a tissue fluid from a human or animal patient, which may be selected from the group consisting of blood, plasma, serum, lymphatic fluid, synovial fluid, cerebrospinal fluid, amniotic fluid, amniotic cord blood, tears, saliva, and nasopharyngeal washes.

In another embodiment of the invention, the assay is a component of a devices that is suitable in fully automated laboratories capable of extracting nucleic acids from a sample (e.g. using the epMotion System of Eppendorf International), optionally capable of reverse transcribing isolated nucleic acids, performing amplification reactions using the assay components described herein and quantitatively and qualitatively detecting nucleic acid targets, e.g. using real-time PCR.

In a further aspect, the present invention relates to a composition comprising any of the above mentioned primers and/or probes. Preferably, the composition comprises also ingredients, e.g. enzymes, buffers and deoxynucleotides necessary for reverse transcription and/or PCR, preferably for qualitative and/or quantitative RT-PCR. The composition may be stored in the refrigerator in a liquid state or deep-frozen in a suitable medium, or it may be lyophilized and reconstituted before use and which may further comprises detectable probes and/or an internal control.

The present invention further provides a kit comprising the assay of the invention and optionally instructions for use.

It is to be understood that while the invention has been described in conjunction with the embodiments described herein, that the foregoing description as well as the examples that follow are intended to illustrate and not limit the scope of the invention. Other aspects, advantages and modifications within the scope of the invention will be apparent to those skilled in the art to which the invention pertains. All patents and publications mentioned herein are incorporated by reference in their entireties.

The following examples are put forth so as to provide those of ordinary skill in the art with a complete disclosure and description of how to make and use the compositions of the invention. The examples are intended as non-limiting examples of the invention. While efforts have been made to ensure accuracy with respect to variables such as amounts, temperature, etc., experimental error and deviations should be taken into account. Unless indicated otherwise, parts are parts by weight, temperature is degrees centigrade, and pressure is at or near atmospheric. All components were obtained commercially unless otherwise indicated.

### EXAMPLES

### Example 1

### Amplification of RSV RNA by Specific Primers and Probes

A real-time PCR assay was run with the following primer-probe combinations, using VR-1540DTM (Human respiratory synctial virus, strain A2) RNA as template.

Primer

| combination | Primer sequence |
|---|---|
| RSV forward | GGCAAATATGGAAACATACGTGAA |
| RSV reverse 1 | GGCACCCATATTGTAAGTGATG |
| RSV reverse 2 | GGCACCCATATTGTTAGTGATG |
| RSV probe 1 | FAM-CTTCACGAGGGCTCCACGTACACAGC-BHQ1 |
| RSV probe 2 | FAM-CTTCACGAAGGCTCCACATACACAGC-BHQ1 |
| RSV probe 3 | FAM-CTTCACGAGGGCTCCACATACACAGC-BHQ1 |

The RSV Taqman probes are labeled with FAM reporter and BHQ-1 quencher. In addition, we have an internal extraction control using a primer/probe set specific for the tobacco mosaic virus (TMV). TMV could be added to samples during RNA extraction. A positive signal detection for TMV means that RNA was successfully extracted from the sample.

A PCR reaction was set-up according to the parameters below. Two sets of reactions were performed. One set was using the RSV primer/probes along. Another set included the TMV primer/probe and TMV RNA.

Rotor-gene Q machine was used to perform the following cycling conditions—55° C. for 5 mins, 60° C. for 5 mins and 65° C. for 5 mins. This is followed by 45 cycles of: 94° C. for 5 s and 60° C. for 40 s. Each reaction was performed in duplicate and the average CT value with standard deviation is listed in the table below.

| RSV RNA | | Ct values for RSV | | Ct values for |
|---|---|---|---|---|
| (ATCC VR-1540D) | | RSV | RSV + TMV | TMV |
| 100 | pg | 25.09 ± 0.06 | 25.26 ± 0.26 | 27.40 ± 0.00 |
| 10 | pg | 28.65 ± 0.19 | 28.67 ± 0.06 | 27.27 ± 0.19 |
| 1 | pg | 31.99 ± 0.04 | 32.24 ± 0.06 | 27.35 ± 0.07 |
| 100 | fg | 35.62 ± 0.29 | 35.94 ± 0.39 | 27.20 ± 0.14 |
| 10 | fg | 39.17 ± 0.07 | 39.35 ± 0.02 | 27.24 ± 0.17 |
| 1 | fg | n.d. | n.d. | 27.40 ± 0.12 |

The test with RSV alone was detected in the green channel, whereas the TMV signal in the duplex assay with TMV EC was detected in the red channel. The RSV signal will be considered positive if the CT value is below 40. The assay is suitable to diagnose both RSV subtype A and subtype B. The double reverse primers and triple probe combination allows for the indiscriminate diagnosis of the presence of RSV from either subtypes. RSV RNA from ATCC VR-955 (a subtype B strain) was also tested and it was found that the assay successfully detected this strain.

This assay is designed as a duplex assay. The advantage of this is it allows inactivated tobacco mosaic virus (TMV) to be added to clinical samples. TMV RNA acts as an internal extraction control to show that RNA is successfully extracted. If the real-time PCR reaction gave negative signals for both RSV and TMV, it means that RNA extraction was unsuccessful and this eliminates a false negative reading.

As shown in the data, the presence of TMV RNA and TMV primer/probes does not affect the sensitivity of the detection, which was 10 fg of total RNA. Moreover, the primers/probes for detecting RSV are designed to target the nucleic acid sequence encoding the “matrix or M protein” from the complete virus genome. Surprisingly, the inclusion of two reverse primers and three probes covers most nucleotide polymorphisms in the RSV M protein.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for the detection of the presence or absence of at least one nucleic acid of Respiratory Syncytial Virus (RSV) in a biological sample, wherein the method comprises:
(a) isolating nucleic acids from the biological sample and optionally performing a reverse transcription step, and
(b) conducting real-time PCR, wherein primer sets that are specific for RSV A and/or RSV B are used, wherein the RSV-specific primer set comprises oligonucleotide sequences set forth in SEQ ID NOs: 1 and 2 or 1 and 3 or complements thereof, or sequences that are at least 95% identical to one or more of SEQ ID NOs: 1, 2 or 3, and
wherein at least one probe specifically binding to a nucleic acid of RSV A and/or RSV B is used, wherein the at least one probe is selected from the oligonucleotides set forth in SEQ ID NOs: 4 to 6, or sequences that are at least 95% identical to one or more of SEQ ID NOs: 4, 5 or 6.

2. The method according to claim 1, wherein the primers and/or probes carry a fluorescent moiety.

3. An in vitro method for the diagnosis of an RSV infection in a subject comprising performing the method according to claim 1.

4. A method for monitoring the treatment of RSV infection, said method comprising performing the method according to claim 3 before treatment with at least one anti-viral drug and during and/or after treatment with said anti-viral drug.

5. A real-time PCR assay for simultaneous detection of at least one nucleic acid of RSV A and/or RSV B in a biological sample comprising primers specifically hybridizing to nucleic acids derived from said RSV A and/or B, using primers and/or probes having oligonucleotide sequences as set forth in claim 1.

6. The assay according to claim 5, wherein the assay is adapted for use in a fully automated laboratory.

7. A composition comprising primers and/or probes as set forth in claim 1.

8. A kit for the detection of RSV A and/or B in a biological sample comprising primers and/or probes having oligonucleotide sequences as set forth in claim 1, further comprising instructions for use.

9. The kit according to claim 8, wherein said kit further comprises enzymes, deoxynucleotides, and/or buffers for performing a reverse transcription step and/or a PCR step.

10. The kit according to claim 8 further comprising reagents for the isolation of nucleic acids from a biological sample.


SEQUENCE LISTING (8 sequences)

SEQ ID NO: 1 - DNA, 24 bp, respiratory syncytial virus
ggcaaatatggaaacatacgtgaa

SEQ ID NO: 2 - DNA, 22 bp, respiratory syncytial virus
ggcacccatattgtaagtgatg

SEQ ID NO: 3 - DNA, 22 bp, respiratory syncytial virus
ggcacccatattgttagtgatg

SEQ ID NO: 4 - DNA, 26 bp, respiratory syncytial virus
cttcacgagggctccacgtacacagc

SEQ ID NO: 5 - DNA, 26 bp, respiratory syncytial virus
cttcacgaaggctccacatacacagc

SEQ ID NO: 6 - DNA, 26 bp, respiratory syncytial virus
cttcacgagggctccacatacacagc

SEQ ID NO: 7 - DNA, 117 bp, respiratory syncytial virus
ggcaaatatggaaacatacgtgaacaaacttcacgaaggctccacatacacagctgctgttcaatacaatgtcctagaaaaagacgatgaccctgcatcacttacaatatgggtgcc

SEQ ID NO: 8 - DNA, 117 bp, respiratory syncytial virus
ggcaaatatggaaacatacgtgaacaagcttcacgaaggctccacatacacagcagctgttcagtacaatgttctagaaaaagatgatgatcctgcatcactaacaatatgggtgcc